(19) United States
(12) Patent Application Publication
(10) Pub. No.: US 2021/0205193 A1
(43) Pub. Date: Jul. 8, 2021

(54) TREATMENT COMPOSITION FOR DYEING

(71) Applicants: Seong Cheol JOO, Siheung-si (KR); SAMIN CHEMICAL CO., LTD, Siheung-si (KR)

(72) Inventors: Seong Cheol JOO, Siheung-si (KR); Nam Mok HYUNG, Anyang-si (KR); Myung Sup SIM, Yongin-si (KR)

(21) Appl. No.: 16/781,522

(22) Filed: Feb. 4, 2020

(30) Foreign Application Priority Data
Jan. 7, 2020 (KR) 10-2020-0001933

(51) Int. Cl. A61K 8/362; A61Q 5/10; A61K 8/46

(52) U.S. Cl. CPC A61K 8/362; A61K 2800/4324; A61K 8/46; A61Q 5/10

(57) ABSTRACT

Provided is a treatment composition for dyeing. The treatment composition for dyeing contains isophorone dimaleate or cystamine dimaleate as an active ingredient. The treatment composition for dyeing contains diamine dimaleate, in particular, isophorone dimaleate or cystamine dimaleate, thereby more effectively covering gray hair, preventing color loss and preventing damage to hair due to dyeing than conventional dyes.

### CROSS-REFERENCE TO RELATED APPLICATION

This application claims priority to and the benefit of Korean Patent Application No. 10-2020-0001933, filed on Jan. 7, 2020, the disclosure of which is incorporated herein by reference in its entirety.

### BACKGROUND OF THE INVENTION

### Field of the Invention

The present invention relates to a treatment composition for dyeing, and more particularly to a treatment composition for dyeing containing diamine dimaleate, in particular, isophorone dimaleate or cystamine dimaleate, which is more capable of effectively covering gray hair, preventing color loss and preventing damage to hair due to dyeing than conventional dyes.

### Description of the Related Art

Recently, as bleaching, dyeing, and heat perms have become popular, the incidence of damaged hair is rapidly increasing. Once damaged, hair cannot be restored. Commercially available hair essences and hair treatments advertise the ability to restore damaged hair, but consumers experience only temporary hair restoration with products using silicones and surfactants.

Meanwhile, hair dyes may be classified into temporary hair dyes, semi-permanent hair dyes and permanent hair dyes depending on the persistence and durability of the dyed color in the hair. Among these, permanent hair dyes are divided into oxidative hair dyes, metal hair dyes and vegetable hair dyes depending on the kind of dye that is used. Most currently used hair dyes are oxidative hair dyes using oxidative dyes such as oxidative dye precursors and amino compounds.

Oxidative hair dyes generally cause an oxidation polymerization reaction between dyes contained in a first agent upon mixing the first agent with a second agent, thus exhibiting a color. In addition, hydrogen peroxide contained in the second agent decomposes the melanin pigment in the hair to thus decolorize the hair and thereby brighten the base (background) color of the hair to thereby render a more vivid color.

Oxidative dyes are small in size and thus can penetrate into the cortex layer of the hair, but they have difficulty penetrating into the hair when the polymer is formed through oxidation. As such, when the size of the molecule is increased through a polymerization reaction before the oxidative dye penetrates into the hair, both the dyeability of the hair and the color persistence after dyeing are deteriorated.

Accordingly, as a result of repeated research on methods of maximizing the dyeing capacity of dyes and color persistence while minimizing hair damage caused by dyeing, the present inventors devised a dyeing treatment composition containing isophorone dimaleate or cystamine dimaleate and found that the treatment composition is capable of more effectively covering gray hair, preventing color loss and preventing damage to hair due to dyeing than conventional dyes. Based on this finding, the present invention has been completed.

### PRIOR ART DOCUMENTS

### Patent Document

(Patent Document 01) Korean Patent No. 10-1294245 (registered on Aug. 1, 2013)
(Patent Document 02) Korean Patent Laid-open No. 10-2018-0127558 (published on Nov. 28, 2018)
(Patent Document 03) Korean Patent No. 10-1800681 (registered on Nov. 17, 2017)

### SUMMARY OF THE INVENTION

Therefore, the present invention has been made in order to solve the problems of the prior art and it is an object of the present invention to provide a treatment composition for dyeing containing diamine dimaleate, in particular, isophorone dimaleate or cystamine dimaleate, which is capable of more effectively covering gray hair, preventing color loss and preventing damage to hair due to dyeing than conventional dyes.

The objects of the present invention are not limited to those described above. The objects of the present invention will be clearly understood from the following description and are capable of being implemented by means defined in the claims and combinations thereof.

In accordance with the present invention, the above and other objects can be accomplished by the provision of a treatment composition for dyeing containing isophorone dimaleate or cystamine dimaleate as an active ingredient.

The treatment composition may have a pH of 3.5 to 4.5.

The isophorone dimaleate or cystamine dimaleate may be present in an amount of 1 to 50% by weight with respect to the total weight of the composition.

Details of other embodiments are incorporated in the Detailed Description of the Invention.

### BRIEF DESCRIPTION OF THE DRAWINGS

The patent or application file contains at least one drawing/photograph executed in color. Copies of this patent or patent application with color drawing(s)/photograph(s) will be provided by the Office upon request and payment of the necessary fee.

The above and other objects, features and other advantages of the present invention will be more clearly understood from the following detailed description taken in conjunction with the accompanying drawings, in which:

FIG. 1 is an image showing the state of the tested hair after using the treatment composition for dyeing according to the invention.

### DETAILED DESCRIPTION OF THE INVENTION

The advantages, features, and methods of accomplishing the same will be clearly understood from the following preferred embodiments with reference to the attached drawings. However, the present invention is not limited to the embodiments, and will be embodied in different forms. The embodiments are suggested only to offer thorough and complete understanding of the disclosed contents and to sufficiently inform those skilled in the art of the technical concept of the present invention.

Terms herein used are used only to illustrate certain embodiments, and should not be construed as limiting the present invention. Singular forms are intended to include plural forms as well, unless the context clearly indicates otherwise.

Unless differently defined, all terms used herein including technical or scientific terms have the same meanings as generally understood by those skilled in the art to which the present invention pertains. In addition, terms identical to those defined in generally used dictionaries should be interpreted as having meanings identical to contextual meanings of the related art, and are not to be interpreted as having ideal or excessively formal meanings unless they are definitely defined in the present specification.

Hereinafter, a treatment composition for dyeing will be described in detail with reference to the attached drawings.

The treatment composition for dyeing according to the present invention includes isophorone dimaleate or cystamine dimaleate as an active ingredient.

In addition, the treatment composition for dyeing according to the present invention may have a pH of 3.5 to 4.5.

In addition, the isophorone dimaleate or cystamine dimaleate in the treatment composition for dyeing according to the present invention may be present in an amount of 1 to 50% by weight with respect to the total content of the composition.

In general, dyeing is based on a principle including penetrating an oxidizing dye into the hair, oxidizing the hair with hydrogen peroxide, polymerization, and rendering a color. The double bond (—C═C—) present in isophorone dimaleate or cystamine dimaleate in the treatment composition for dyeing according to the present invention facilitates polymerization to form a polymer having a higher molecular weight than a conventional oxidized polymer, thus obtaining more vivid and deeper colors and restoring a cleaved —S—S— bond.

That is, in general, dyeing is based on a principle in which an oxidizing dye (para-phenylenediamine, etc.) reacts with hydrogen peroxide to produce an intermediate precursor and then a final polymer, as shown in the following Chemical Reaction Scheme.

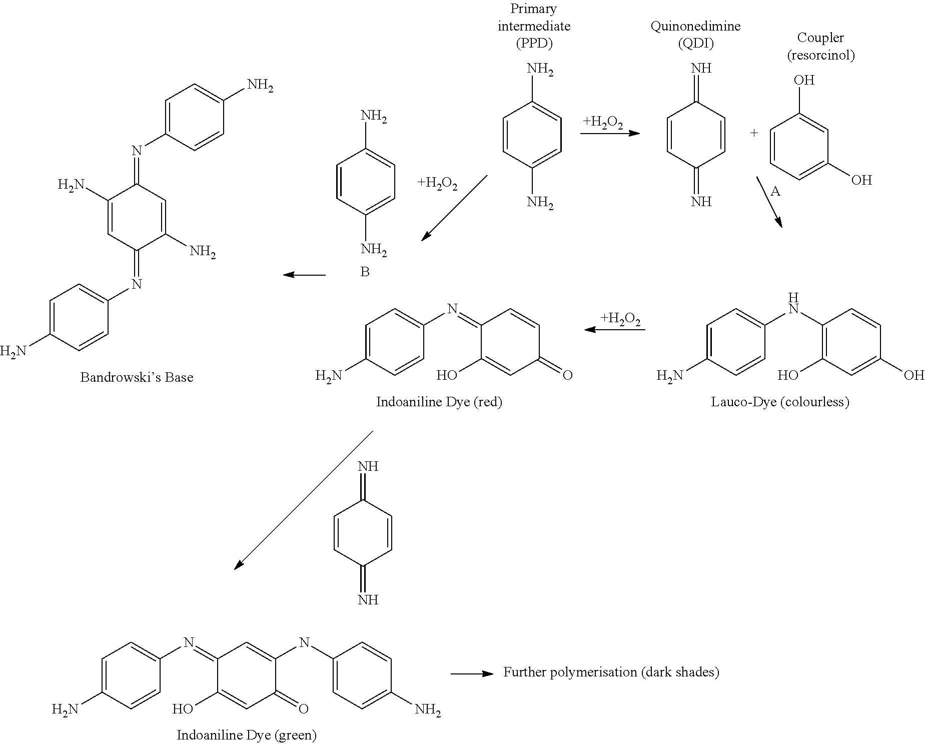

As depicted in the chemical reaction scheme above, the polymer (dark shades) as a final product reacts with the double bond of isophorone dimaleate or cystamine dimaleate according to the present invention to produce a polymer having a higher molecular weight and thereby obtain a more vivid and deeper dyeing effect.

In addition, the double bond of isophorone dimaleate or cystamine dimaleate in the treatment composition for dyeing according to the present invention exerts an effect of rebonding the cleaved —S—S— bond of damaged hair, thus exerting an excellent effect of restoring damaged hair.

In general, frequent dyeing, discoloration and perm procedures result in cleavage of the disulfide bond (—S—S—) of the hair, causing damage to the hair and an increase in thiol (—SH) residue on the damaged hair. Isophorone dimaleate or cystamine dimaleate in the treatment composition for dyeing according to the present invention can restore the disulfide bond of damaged hair as described above, thereby recovering damaged hair.

Principle of Damaged Hair Recovery by Isophorone Dimaleate

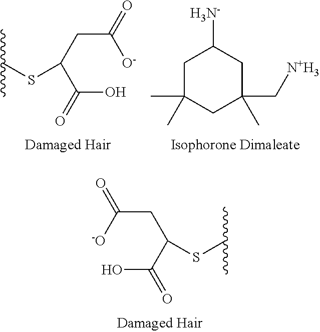

Principle of Damaged Hair Recovery by Cystamine Dimaleate

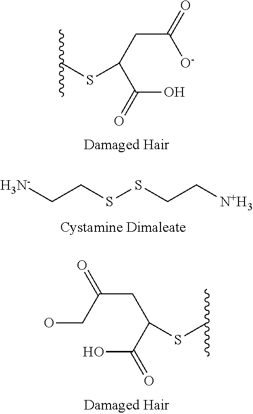

Hereinafter, the specific experimental example for the treatment composition for dyeing according to the present invention will be described in more detail.

The treatment composition for dyeing was prepared in accordance with the following ingredient composition table and weight ratio.

### 1. Experimental Example 1

| Ingredient composition table | %(wt/wt) |
|---|---|
| Isophorone dimaleate | 10.0 |
| Cationic surfactant | 10.0 |
| Cationic guar gum | 2.0 |
| Hydroxypropyl chitosan | 0.5 |
| Perfume | 0.2 |
| Monoethanolamine | 0.5 |
| Purified water | Balance |
| Total | 100.0 |

### pH of Experimental Example 1=4.0

### 2. Experimental Example 1

| Ingredient composition table | %(wt/wt) |
|---|---|
| Cystamine dimaleate | 10.0 |
| Cationic surfactant | 10.0 |
| Cationic guar gum | 2.0 |
| Hydroxypropyl chitosan | 0.5 |
| Perfume | 0.2 |
| Monoethanolamine | 1.0 |
| Purified water | Balance |
| Total | 100.0 |

### pH of Experimental Example 2=4.2

### 3. Experimental Method

An appropriate amount of the treatment composition obtained by mixing at the weight ratio of Experimental Examples 1 and 2 was applied to the bleached hair, then allowed to stand for 10 minutes, washed with lukewarm water and dried with a dryer, and compared with the untreated hair.

### 4. Use of Hair Dye

The bleached hair may be dyed with a commercially available dark brown (3N) dye, or with a hair dye prepared as follows.

The hair dye may consist of a first agent containing an oxidizing dye and an alkalizing agent, and a second agent, which is mixed with the first agent and contains an oxidizing agent.

The oxidizing dye performs a hair dyeing action, the alkaline agent swells the hair so that the oxidizing dye can penetrate into the hair, and the oxidizing agent oxidizes the first agent so that the oxidizing dye is fixed to the hair.

In the present invention, the first agent is purified water (DI water), 2-amino-5-nitrophenol, polyquaternium-10, polysorbate 80, tetrasodium EDTA (EDTA-4Na), p-phenylenediamine, m-aminophenol, ethanolamine thioglycolate, PEG-60 castor oil, a perfume, dimethicone, oleic acid, polyoxyethylene oleyl ether, behenyl alcohol, sodium silicate, myristyl alcohol, monoethanolamine, cyclodextrin, allantoin, an aloe extract, hydroxypropyl chitosan, and a Houttuynia cordata Thunb. extract.

In addition, the first agent in the natural hair dye composition according to the present invention contains 80 to 85 parts by weight of purified water (DI water), 1.5 to 2.5 parts by weight of 2-amino-5-nitrophenol, 1.5 to 2.0 parts by weight of polyquaternium-10, 1 to 3 parts by weight of polysorbate 80, 0.02 to 0.04 parts by weight of tetrasodium EDTA (EDTA-4Na), 0.8 to 1.2 parts by weight of p-phenylenediamine, 1.5 to 2.5 parts by weight of m-aminophenol, 2.2 to 3.2 parts by weight of ethanolamine thioglycolate, 1.2 to 1.5 parts by weight of PIG-60 castor oil, 0.2 to 0.5 parts by weight of a perfume, 0.005 to 0.01 parts by weight of dimethicone, 0.3 to 0.9 parts by weight of oleic acid, 3 to 5 parts by weight of polyoxyethylene oleyl ether, 2.5 to 4.5 parts by weight of behenyl alcohol, 0.4 to 0.8 parts by weight of sodium silicate, 1 to 3 parts by weight of myristyl alcohol, 1.1 to 1.3 parts by weight of monoethanolamine, 0.05 to 0.09 parts by weight of cyclodextrin, 0.007 to 0.013 parts by weight of allantoin, 0.05 to 0.07 parts by weight of an aloe extract, 0.005 to 0.015 parts by weight of hydroxypropyl chitosan and 1 to 2 parts by weight of a Houttuynia cordata Thunb. extract.

The Houttuynia cordata Thunb. extract is prepared by extracting Houttuynia cordata Thunb. Houttuynia cordata Thunb. is a terrestrial part of Houttuynia cordata Thunberg which is a plant of the Saururaceae family, and inhabits Southeast Asia, especially Japan, Korea and the like. Houttuynia cordata Thunb. is also called “ten-drug” because it has ten medicinal effects, and is so named because the stem resembles a sweet potato leaf and the raw leaves emit a bad fishy smell when touched. Houttuynia cordata Thunb. can be used for medicinal and edible foods, is classified as an animal/plant that can be used only in a minimum amount of a minor ingredient in the classification of food raw material in the Health Functional Food Code is well-known to have cardiac, diuretic, antibacterial, detoxification and anticancer effects, and is used as a cosmetic and health functional food to contribute to detoxification and beauty in private.

The Houttuynia cordata Thunb. extract herein used may be the Houttuynia cordata Thunb. extract prepared by the following preparation method.

First, in order to prepare the Houttuynia cordata Thunb. extract, Houttuynia cordata Thunb. is prepared and then washed.

Then, the washed Houttuynia cordata Thunb. may be pulverized to a certain particle size.

In this step, the Houttuynia cordata Thunb. may be pulverized to a predetermined particle size so that the active ingredient of the Houttuynia cordata Thunb. can be sufficiently extracted. In this step, the Houttuynia cordata Thunb. may be pulverized to adjust the particle size to fall within the range of 500 to 2,500 μm. In this step, when the particle size of the Houttuynia cordata Thunb. is smaller than 500 μm, it takes a long pulverization time, and the particles are excessively small, thus causing agglomeration between the particles in subsequent processing. When the particle size of the Houttuynia cordata Thunb. is larger than 2,500 μm, there may be a problem in that it is difficult to effectively extract useful ingredients from the Houttuynia cordata Thunb.

Subsequently, the pulverized Houttuynia cordata Thunb. is sonicated in a low-temperature reduced-pressure atmosphere and is then extracted to prepare a Houttuynia cordata Thunb.extract mixture.

In this step, the pulverized Houttuynia cordata Thunb. is mixed with a solvent to prepare a mixed solution, and the mixed solution is injected into an ultrasonic extractor and then ultrasonic wave was applied to the mixed solution to prepare a Houttuynia cordata Thunb. extract mixture. The solvent may be mixed in an amount of 1,000 to 2,000 parts by weight with respect to 100 parts by weight of the pulverized Houttuynia cordata thumb, and the solvent may include any one or more solvents selected from water or C1-C4 alcohols having 1 to 4 carbon atoms (C1-C4 alcohols).

In addition, in this step, the extraction may be carried out by applying ultrasonic wave to the mixed solution at a temperature of 20 to 25° C. and a pressure of 0.1 to 0.5 kgf/cm² at a power of 100 to 200 watt at a vibration frequency of 30 to 50 kHz for 30 to 60 minutes.

Subsequently, solids such as crushed Houttuynia cordata thumb are removed from the Houttuynia cordata thumb extract mixture, and then the residue is aged at a temperature of 10 to 20° C. for 1 to 3 hours to prepare a Houttuynia cordata thumb extract.

In this step, the removal of solid crushed Houttuynia cordata thumb from the Houttuynia cordata thumb extract mixture may be carried out by separating and removing the solids using a known filtering net such as a sieve.

In the present invention, the second agent contains purified water (DI water), hydrogen peroxide, sodium pyrophosphate, methyl paraben (MP), triethanolamine, etidronic acid, cetearyl alcohol, glyceryl stearate, lanolin alcohol, a perfume, Ceareareth-20, PEG-120 methyl glucose dioleate, behenyl alcohol, allantoin and a Pu-erh tea extract.

In addition, in the present invention, the second agent may contain 75 to 80 parts by weight of purified water (DI Water), 10 to 20 parts by weight of hydrogen peroxide, 0.5 to 1.5 parts by weight of sodium pyrophosphate, 0.8 to 1.2 parts by weight of methyl paraben (MP), 0.5 to 1.5 parts by weight of triethanolamine, 1 to 2 parts by weight of etidronic acid, 1 to 3 parts by weight of cetearyl alcohol, 0.1 to 0.5 parts by weight of glyceryl stearate, 1.2 to 2.2 parts by weight of lanolin alcohol, 0.3 to 0.5 parts by weight of a perfume, 0.15 to 0.35 parts by weight of Ceteareth-20, 0.25 to 0.45 parts by weight of PEG-120 methyl glucose dioleate and 0.1 to 0.3 parts by weight of allantoin.

### 5. Experimental Table

| | | | Mix ratio | | |
|---|---|---|---|---|---|
| | | | between first | | |
| | First | Second | agent and | Intermediate | Standing |
| Item | agent | agent | second agent | applying | time |
| A | 5 g | 5 g | 1:1 | X | 20 min |
| B | 5 g | 5 g | 1:1 | Experimental | 10 min, |
| | | | | Example 1 solution | applying, |
| | | | | 0.5 g | 10 min |
| C | 5 g | 5 g | 1:1 | Experimental | 10 min, |
| | | | | Example 2 solution | applying, |
| | | | | 0.5 g | 10 min |

In the Experimental Table above, Section A represents a conventional dyeing method and shows the result obtained by applying a hair dye to the hair, allowing the dye to stand for 20 minutes, washing and drying, Section B shows the result obtained after applying a hair dye to the hair, allowing the dye to stand for 10 minutes, applying the solution according to Experimental Example 1 to the hair, allowing the solution to stand for 10 minutes, washing and drying, and Section C shows the result obtained after applying a hair dye to the hair, allowing the dye to stand for 10 minutes, applying the solution according to Experimental Example 2 to the hair, allowing the solution to stand for 10 minutes, washing and drying.

### 6. Experimental Results

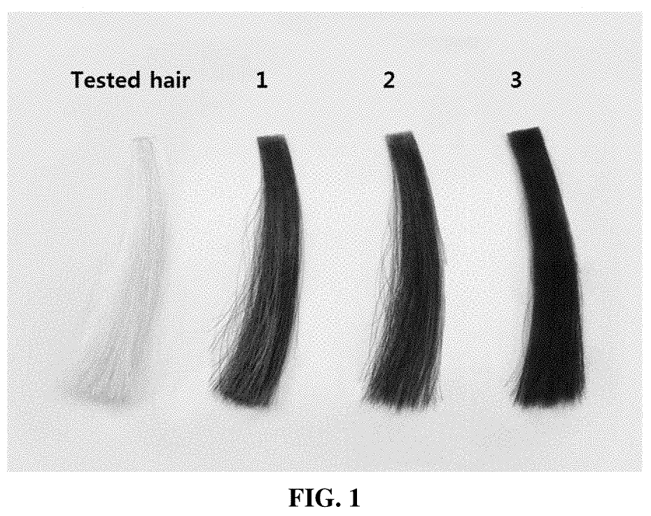

FIG. 1 is an image showing the state of the tested hair after using the treatment composition for dyeing according to the invention.

As can be seen from FIG. 1, it can be seen that the color of the hair deepens in the order of Section A<Section B<Section C.

In addition, as a result of comparing the color of the dyed hair every day after shampooing for 10 days, the color loss of the hair of Sections B and C is much less than that of the hair of Section A.

In addition, the result of sensory experiments evaluated by 10 panelists showed that the degree of damage to hair generated during dyeing was the most severe in Section A and that there was almost no damage in Section B and Section C, which were similar to each other.

### 7. Review

P-phenylenediamine, which is used as a dye for hair dyes, is oxidized by hydrogen peroxide, as a second agent for dyeing to produce a polymer. When the polymer is treated with the isophorone dimaleate or cystamine dimaleate in the treatment composition for dyeing according to the present invention, the polymer reacts with —C═C— double bonds present in both substances, resulting in a higher degree of polymerization and thus a higher-molecular-weight polymer and a deeper color.

In addition, that the color of Section C was deeper than Section B is considered to be due to the increase in the lateral polymerization degree for Section B and to be due to the steric polymerization by the non-covalent electron pair of —S—S-bond for Section C.

Degree of Polymerization

Section B and Section C have less color loss and better color retention than Section A because Section B and Section C higher molecular weights of oxidized polymers and thus have more difficulty escaping from hair through cuticles. The damage after dyeing of Section B and Section C is less than that of Section A because the diamine dimaleate restores —S—S— bonds cleaved during dyeing.

The treatment composition for dyeing according to the present invention is capable of rendering more vivid color, improving color retention and preventing hair damage during dyeing by using a treatment containing isophorone dimaleate or cystamine dimaleate as a main ingredient for dyeing.

As apparent from the foregoing, the treatment composition for dyeing contains diamine dimaleate, in particular, isophorone dimaleate or cystamine dimaleate, thereby effectively covering gray hair, preventing color loss and preventing damage to hair due to dyeing compared to conventional dyes.

The effects of the present invention are not limited to those mentioned above. It should be understood that the effects of the present invention include a variety of effects that can be inferred from the foregoing description of the present invention.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A treatment composition for dyeing comprising isophorone dimaleate or cystamine dimaleate as an active ingredient.

2. The treatment composition according to claim 1, wherein the treatment composition has a pH of 3.5 to 4.5.

3. The treatment composition according to claim 2, wherein the isophorone dimaleate or cystamine dimaleate is present in an amount of 1 to 50% by weight with respect to the total weight of the composition.
